(19) United States
(12) Patent Application Publication
(10) Pub. No.: US 2014/0193464 A1
(43) Pub. Date: Jul. 10, 2014

(54) EFFECTS OF PROBIOTICS ON HUMANS AND ANIMALS UNDER ENVIRONMENTAL OR BIOLOGICAL CHANGES

(71) Applicant: Jhy-Jhu Lin, Potomac, MD (US)

(72) Inventor: Jhy-Jhu Lin, Potomac, MD (US)

(73) Assignee: IMAGILIN TECHNOLOGY, LLC, Frederick, MD (US)

(21) Appl. No.: 13/736,455

(22) Filed: Jan. 8, 2013

(51) Int. Cl. A61K 36/00; A61K 35/74

(52) U.S. Cl. CPC A61K 36/00; A61K 35/744
USPC 424/400; 424/93.1; 424/93.45; 424/93.4; 424/93.44; 424/93.51

(57) ABSTRACT

A dry, stable and viable probiotic composition comprising, a probiotic microorganism and a dried plant powder, with the proviso that said composition is not a blended mixture of at least one biologically pure Pediococcus acidilactici probiotic culture and dried tomato powder at a weight ratio of 1:4 encapsulated in an effective amount in a gelatin capsule.

### BACKGROUND OF THE INVENTION

1. Field of the Invention

Probiotics are beneficial microorganisms naturally existing in gastrointestinal (GI) tracts of humans and animals. Probiotics are described to have health benefits when administered the right amounts of live microorganisms into humans and animals. Probiotics are widely applied as nutritional supplements in animals and humans. For example, yeast is used as a nutrient supplement for livestock, and yogurt with lactic acid bacteria Lactobacillus and/or Bifidobacterium is commonly used. No toxic effects are described when administered probiotics as nutrition supplements into humans and animals.

2. Description of the Related Art

In order to have the maximum effects of probiotics on animals and humans, one has to administrate live bacteria to reach gastrointestinal tracts for multiplication (Kailasapatha and Chin 2000). Lactobacillus spp and Bifidobacterium spp are two most commonly probiotics described in scientific literature and in commercial products. Both Lactobacillus spp and Bifidobacterium spp are facultative anaerobic bacteria. Most species (or strains) of Lactobacillus and Bifidobacterium are sensitive to the exposure of oxygen (Gomes et al, 1995: Talwalkar and Kailasapathy, 2004) and high temperature. It is difficult to maintain the viability of Lactobacillus and Bifidobacterium at room temperature under consistent open and closure operations. Therefore, variable results are often described, especially for commercially available products that are required to have long term storage and shipping in various temperature (Tuomola et al, 2001).

Vegetables and fruits are the main sources of fibers, vitamins, natural antioxidants and minerals for humans and animals. For examples, tomato and cabbage were described as the natural source of vitamin C (Clayton and Borden, 1942). More important, the safety of vegetables and fruits has been well accepted. Recently, natural berries like Acerola that is described to be rich in ascorbic acid and polyphenols. The high content in vitamin C (695 a 4827 mg/100 g) make Acerola as the preferred choice of natural vitamin C (Mezadri et al, 2006). In addition, recent study in rats demonstrated that Acerola is safe to be as food supplement for human consumption (Hanamura and Aoki, 2008). The combination of probiotics with fresh vegetables or fruits will offer benefits from probiotics and fresh vegetables or, fruits. However, fresh vegetables and fresh fruits are the natural nutritional sources for microorganisms to multiply. The replication of microorganisms in fresh vegetables and fruits not only changes the nutritional compositions of vegetables or fruits but also creates toxic compounds which either are secreted from microorganisms or generated as the side products from the replication of microorganisms or decomposition of vegetables or fruits. Whenever probiotics start to be active, it becomes difficult to keep them alive for long term storage, especially at room temperature.

Research scientists and commercial companies have developed different dried process to preserve the vegetables and fruits. The main challenges of dried vegetables or fruits are to maintain nutrition and flavors of vegetables and fruits. It is critical during the drying process to maintain the minerals, vitamins, carbohydrates, proteins, and antioxidants of vegetables or fruits as much as possible, especially for dried vegetable or fruit powders. In addition, during the drying process and storage, the loss of nutrition and flavors of dried vegetables or fruit powders has to be minimal to have the nutritional benefits as fresh vegetables and fruits.

It is well known that when one mixed probiotics with minerals or other animal feed additives, the viability of the probiotics decreased significantly. Single 1 s encapsulation of the mixture of probiotics with vitamin and mineral supplements within a gelatin capsule resulted in the loss of more than 99.79% of viability of the probiotics (Zimmer, 1996, U.S. Pat. No. 5,501,857). This creates a major challenge: to add probiotics to dried vegetable or fruit powders which required keeping the minerals, vitamins, antioxidants and flavors of the powders while maintaining the viability of the probiotics.

Humans and animals are vulnerable to become ill under environmental changes, such as separation from family, travel, stay in hotel or boarding facilities, or temperature or by biological changes such as aging, diet changes, pathogens or parasites infection, or antibiotic treatment. Such environmental changes often show an increase in the release of hormones. The most important of these hormones is cortisol from the adrenal cortex. Cortisol causes a suppression of the inflammatory response (Roberts et al. 2006). Prolonged increased levels of cortisol cause a decreased ability to mount an immune response (Roberts et al. 2006). A suppressed immune system impacts the host in many ways, for example, there is a weakened ability to engulf invading bacteria. Elevated blood cortisol affects the fluidity of macrophage membranes; macrophage ability to kill ingested pathogens is reduced when the immune system is suppressed (Mayo Clinic 2006). Medical doctors and veterinarians often treat gastrointestinal (GI) diseases with weeks of antibiotic or steroid therapy. Prolonged use of broad-spectrum antimicrobials, however, can disrupt the populations of beneficial microorganisms in human and animal GI tracts, and cause side effects of digestive disorders. Application of steroids in human and animal often caused disruption of natural hormones, cardiovascular disease, liver disease, and skin disease. Usage of probiotics to negate impacts changes caused by environmental or biological changes of humans or animals decreases the reliance medical doctors and veterinarians have on antibiotics and steroids.

The foregoing examples of the related art and limitations related therewith are intended to be illustrative and not exclusive. Other limitations of the related art will become apparent to those of skill in the art upon a reading of the specification.

It is disclosed in U.S. application Ser. No. 12/386,285, filed Apr. 16, 2009 that the deleterious effects caused by environmental or biological changes in human or animals can be ameliorated by feeding a human or animal in need of such amelioration a probiotic composition comprising a blended mixture of at least one biologically pure Pediococcus acidilactici probiotic culture and dried tomato powder at a weight ratio of 1:4; encapsulated in an effective amount in a gelatin capsule.

### SUMMARY OF THE INVENTION

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

Embodiments include compositions comprised of probiotics and at least one dried plant powder. Embodiments include probiotic microorganisms such as Pediococcus, Bifidobacterium, Bacteroides, Propionibacterium, Streptococcus, Enterococcus, Lactococcus, Lactobacillus, and Saccharomyces. Embodiments include dried plant powders from vegetables, fruits, cereals and herbs. In other embodiments compositions are encapsulated in gelatin capsules.

Additional embodiments include the process of ameliorating effects caused by environmental or biological changes in human or animals comprising the step of: feeding the human or animal in need of such amelioration a probiotic composition comprising a viable encapsulated probiotic microbe. Environmental change is defined as stays in boarding facilities, travel, temperature changes, new and/or detrimental changes in the immediate human or animal area or scene. Biological change is defined as aging of humans and animals, infection by pathogens or parasites, chronic physiological changes, and changes in established bodily functions.

Still other embodiments include the process of ameliorating effects caused by environmental or biological changes in human or animals comprising the step: feeding the human or animal in need of amelioration a probiotic composition comprising: a probiotic microorganism and a dried plant powder.

In addition to the exemplary aspects and embodiments described above, further aspects and embodiments will become apparent by reference to the drawings and by study of the following descriptions.

### DETAILED DESCRIPTION OF THE INVENTION

The present invention is predicated on the surprising discovery that the deleterious effects caused by environmental or biological changes in human or animals can also be ameliorated by feeding a human or animal in need of such amelioration a probiotic composition comprising a blended mixture of at least one biologically pure Pediococcus acidilactici probiotic culture and any vegetable powder at weight ratios of other than 1:4; preferably encapsulated in an effective amount in a gelatin capsule.

Pediococcus acidilactici fermentation cultures were blended with tomato spray dried powders at the weight ratio of 1:4. Encapsulated 500 mg mixtures of P. acidilactici and dried tomato powders into a size O gelatin capsules by manual capsule filling apparatus or automatic capsule filling machine. The gelatin capsules were stored at room temperature. Capsules were stored at room temperature and were taken from day 0, 1, 3 and 6 month after being stored at room temperature. After separation of the capsules, 1 gm of the mixtures of P. acidilactici and tomato dried powders were re-suspended up to 10 ml sterilized saline buffer. A series of standard dilutions were performed, and 100 ul of 10⁷ or 10⁸ dilutions were sprayed onto MRS (de Man, Rogosa and Sharpe) agar plates. The plates were incubated at 45° C. until colonies were observed for quantitative analysis. The bacterial viability is shown in Table 1.

TABLE 1
Stability of encapsulation of Pediococcus acidilactici
fermentation cultures with tomato dried powders
in a gelatin capsule at room temperature

| Time after Manufacture, | Number of bacteria | |
|---|---|---|
| Months | CFU/gm | % of survival |
| 0 | 2.0 × 10¹⁰ | 100 |
| 1 | 1.92 × 10¹⁰ | 96 |
| 3 | 1.94 × 10¹⁰ | 94 |
| 6 | 1.76 × 10¹⁰ | 88 |

Table 1 shows that a culture of probiotic microorganisms blended with spray dried tomato powder retains substantial viability for at least six months.

Clients with digestive disorders were volunteers from China, Taiwan and USA, and referred by the local clinics to take NutriTot™, a probiotic composition comprising P. acidilactici fermentation cultures in dried vegetable powders such as tomato, carrot, sweet potato, cabbage, spinach or broccoli or fruit powders such as lemon, peach, strawberry, or apple in a gelatin capsule. Administration of NutriTot™: Children<10 years old, half capsule per day; Adult and Children>10 years old, 1 capsule per day Double amounts of NutriTot™ when the symptoms are moderate severe to severe or severe. The results are shown in Table 2.

TABLE 2
Effects of P. acidilactici fermentation cultures in dried vegetable powders or
fruit powders on human with digestive disorders

| | | | CONDITIONS | | | |
|---|---|---|---|---|---|---|
| | | | PRIOR TO | | | |
| | | | FEEDING | | | |
| | | | PROBIOTICS | | EFFECTS OF | |
| | | | .D, O, V, C, F, LA.¹ | | TREATMENT | |
| | | | (Severe: ++++, | | (Excellent: ++++, | |
| | | | Moderate to | | Very Good: +++, | |
| | | | Severe: +++, | | Good: ++ | |
| AGE | | | Moderate: ++, | | Slight | |
| (yrs | WEIGHT | | Mild: +, | TREATMENT | Improvement: + | |
| old) | (Kg) | SEX | Normal: 0) | (DAYS) | No effects: 0)² | COMMENTS |
| 74 | 44 | F | +++ | 14 | +++++ | Appetite becomes |
| | | | LA, O | | | normal in two days |
| 6 | 20 | M | +++ | 14 | +++++ | Appetite becomes |
| | | | LA | | | normal in two days |
| 75 | 52 | M | ++++ | 21 | +++++ | Appetite becomes |
| | | | C, LA | | | normal in two days, |
| | | | | | | Improved |
| | | | | | | constipation started at |
| | | | | | | week 2 |
| 65 | 60 | M | ++++ C | 14 | +++++ | Normal bowel |
| | | | | | | movement in 3 days, |
| | | | | | | no more constipation |
| 74 | 66 | M | +++ C | 3 | +++++ | No more constipation |
| 30 | 55 | F | ++++ D | 14 | +++++ | Stop diarrhea in two |
| | | | | | | days, stool showed as |
| | | | | | | banana shape |
| 23 | 50 | F | ++++ D | 2 | +++++ | Diarrhea stop |
| 41 | 75 | M | ++++ | 14 | ++++ | Increase appetite in |
| | | | O, F, LA | | | first week, decrease |
| | | | | | | Flatulence in second |
| | | | | | | week |
| 19 | 65 | M | +++ D | 5 | +++++ | Stop diarrhea |
| 39 | 52 | F | +++ V | 2 | +++++ | Stop vomiting |
| 84 | 84 | M | ++++ C | 30 | +++++ | Improved constipation |
| | | | | | | in first week |
| 7 | 20 | M | +++++ | 3 | +++++ | Appetite fully |
| | | | LA | | | recovered in two days |
| 55 | 158 | F | ++++ D | 180 | +++++ | Stop diarrhea |
| 68 | 174 | M | ++ C | 180 | +++++ | Normal bowel |
| | | | | | | movement, energetic. |

¹D: Diarrhea, O: bad mouth or body odor, V: Vomiting, C: Constipation, F: Flatulence, LA: Loss of Appetite
²Symptoms improvement: Excellent: <2 days, Very Good: between 2 days and 4 days, Good: between 5 days and 14 days, Slight Improvement: between 15 days and 30 days No Improvement >1 month.

Table 2 shows the beneficial effect on human with biological changes in digestive conditions of feeding with probiotics with dried vegetative powder or dried fruit powder. The beneficial effects often observed within a week or less like two days after NutriTot™ applications.

Dogs experience digestive disorders and/or discomforts when kept in boarding facilities for a variety of reasons: the dogs must sleep in a new places, their diet may have changed, they are exposed to other dogs (many of which are barking very loudly), and they do not see the people they normally see every day. These digestive disorders and/or discomfort result from either fear or anxiety (Casey 2002). Fear is defined as an emotional response to a potentially dangerous stimulus, whereas anxiety is the emotional response to a stimulus that predicts a potentially dangerous or unpredictable environment (Casey 2002). Thus, anxiety is the anticipation of harm, whether real or imaginary (Frank et al. 2006).

Two veterinary hospitals in Maryland participated in the study of probiotics on dogs staying the boarding facility. A clinic in Frederick participated from December 2007 through February 2008. A clinic in Baltimore participated from January through March 2008. One hundred fifty-four dogs participated in the study, 83 dogs were in the treatment group, and 71 dogs were in the untreated group, which served as a control. Canines in the test group were fed one capsule of probiotics once a day if less than 50 lbs, two capsules if weighing 50 lbs or more; technicians administering probiotics orally to study participants wrote their initials on study forms indicating the animal had received their daily dosage. Each capsule of probiotics contained an estimated colony forming units (CFU) of Pediococcus acidilactici and Saccharomyces boulardii from fermented culture.

Veterinary records of all study participants were reviewed for health history and past medications, to track for any potential underlying causes of diarrhea in study participants (i.e. history of frequent diarrhea, usage of antibiotics). Study forms to record each dog's information were used to collect the results.

Differences between treated and untreated group's stool consistency were recorded for all dogs entering the study after the study form was updated. Because of low incidence of diarrhea in study participants, GID scoring, as described below, was used as an alternative measure of intestinal distress. Dogs received one GID score for each bowel movement that was analyzed. A single digit score was made for each stool sample that was analyzed, and scores were recorded on study forms by veterinary staff and described as the followings: normal stool, a GID score of ‘1,’ soft/unformed stool, a GID score of ‘2, and diarrhea, a GID score of ‘3’. Higher percentages indicate greater incidence of diarrhea and soft/unformed stool. Values illustrated above were determined by the following formula: (total GID score/total bowel movements)×100=% affected bowel movements. The results are shown in Table 3.

TABLE 3
Effects of probiotics on the dogs staying in boarding facility.

| Parameter | Untreated | Treated | p value (t test) |
|---|---|---|---|
| Number of dogs | 71 | 83 | |
| Total bowel movements | 132 | 233 | |
| GID score | 96 | 29 | |
| Soft stool† | 36 | 7 | <0.0001 |
| Diarrhea† | 7 | 5 | 0.1 |
| Vomiting† | 1 | 0 | 0.18 |

†Data is the incidence of parameter.

Table 3 shows the beneficial effect of probiotics on bowel movements in dogs under the environmental changes of confinement in a boarding facility; namely, greater incidence of diarrhea and soft stool in boarded untreated dogs than treated dogs.

When fish are transported from a fish breeder to a delivery site they are confined within a plastic bag consisting of water and oxygen, many instances for more than twenty four hours. During transport, the water in these closed containers may become oxygen-depleted, and may accumulate excessive carbon dioxide and consequently undergo a reduction in pH (Cole 1999). Metabolic activity may also lead to elevated ammonia levels in the water, which can be damaging to fish health, or become lethal in extreme cases (Cole 1999).

Disease is a major problem for the fish farming industry (Gram et al. 2003). Although vaccines to fish pathogens are being developed and marketed, they generally cannot be used as a universal disease control measure in aquaculture (Gram et al. 2003). Juvenile fish are not fully immunocompetent and do not always respond to vaccination (Gram et al. 2003).

The goldfish (Carassius auratus) belongs to a class of fish called teleost which literally means bony fish. In a study by Ahilan et al. in 2004 qualitative analysis of gut flora of juvenile goldfish was conducted and the presumptively identified microbes were Micrococcaceae, Arthrobacter, Lactobacillus, Bacillus, Vibrio, Pseudomonas, Acinelobacter, Enterobacteriaceae and Alcaligenes. We apply Pediococcus acidilactici to determine the extent to which probiotics influence goldfish under induced physical and/or biological changes. Goldfish were chosen for this study due to the number of mortalities that occur during their handling process, and their vulnerability to infectious diseases along with the ease in obtaining a large number for the study along with the popularity of the fish.

Goldfish (Carassius auratus) were obtained from a pet distributor. The fish were fed the probiotic supplemented diet for three days. On the fourth day fecal samples was collected. The physical change was induced by placing the fish in a closed plastic container with water and oxygen for three hours. The fish were continuously fed for two weeks with or without the probiotic supplemented feed. During the two week period the survival rate was observed. For the treatment group, the feed composed of ground up fish feed (Omega One marine pellets, Sitka, et al.), alginic acid (Acros Organics, N.J.), and 0.1% of the lyophilized probiotic by total weight of the feed mixture. A minimal amount of water was added to the mixture to homogenize. The mixture was then extruded through an empty syringe to obtain thin, long strands of feed. The strands were washed in 0.25M CaCl₂, followed by a rinse with de-ionized water. The feed was then cut into appropriate sizes to allow for fish intake. The amount of probiotic in the feed once fully processed corresponds to approximately 1.7.times.10.sup.8 cfu/gram of feed. The feed for the control group was processed similarly with the exclusion of the probiotics. Table 4 shows the results.

TABLE 4
Effects of Pediococcus acidilactici on mortality
of gold fish treated with environmental changes.

| | | Number of gold fish | |
|---|---|---|---|
| | Total numbers of | dies after physical | |
| Treatment | gold fish | changes | % of mortality |
| Control | 21 | 10 | 47.6% |
| P. acidilactici | 21 | 4 | 19.0% |

Table 4 shows the effect of probiotics on mortality in fish which went through the environmental changes of confinement, crowding, and elevated temperature.

Goldfish infected by Ichthypothirius multifilis were obtained from a pet shop. Using the same conditions as in Table 4, the fish were fed the probiotic supplemented diet for three days. On the fourth day fecal sample was collected. The physical change was induced by placing the fish in a closed plastic container with water and oxygen for three hours. The fish were continuously fed for two weeks with or without the probiotic supplemented feed. During the two week period the survival rate was observed. Table 5 shows the results.

TABLE 5
Effects of P. acidilactici on mortality of gold
fish infected with Ichthypothirius multifilis and
treated with environmental changes as in Table 4.

| | Total numbers of | Number of gold fish | |
|---|---|---|---|
| Treatment | gold fish | dead | % of mortality |
| Control | 20 | 12 | 60% |
| P. acidilactici | 20 | 5 | 40% |

Table 5 shows the effect of probiotics on preventing death in biological changed fish—infected with a parasite, and treated with environmental changes as in Table 4.

Dogs suffering from digestive orders chronically-biological changes, and diagnosed as having Inflammatory Bowel Diseases (IBD) symptom, were treated with mixtures of Pediococcus acidilactici and Saccharomyces boulardii as 1 capsule for body weight (BW) under 5 kg, 2 capsules for BW under 5-15 kg, 3 capsules for BW 15-30 kg, and 4 capsules for BW over 30 kg. The field evaluation was performed at Dakutari Animal Hospital Hiroo Central Hospital (Tokyo, Japan). Table 6 shows the results.

TABLE 6
Effects of probiotics on does suffering with Inflammatory Bowel Diseases (IBD)

| | | | | Symptoms | | | |
|---|---|---|---|---|---|---|---|
| | | | | at small | | | |
| | | | Condition | intestines | Steroid | | |
| | | | prior to | (SI), large | treatment | | |
| | | | feeding | intestines | During | Days of | |
| | | Sex | probiotics | (LI), other | probiotic | probiotic | Effects of |
| Ages | Weight | (M, F, C, S)¹ | (D, V, C, F, LA)² | organs | treatment | treatment | probiotics |
| 12 | 4.7 | C | ++ D | IBD, LI, | + | 3 | Changed |
| | | | | Stomatitis | | | to |
| | | | | | | | probiotics |
| | | | | | | | only. |
| | | | | | | | Improving, |
| | | | | | | | looks |
| | | | | | | | good |
| 6 | 4.6 | C | ++ | IBD, LI, | + | 3 | Changed |
| | | | D, LA | Stomatitis | | | to |
| | | | | +++ | | | probiotics |
| | | | | | | | only, |
| | | | | | | | Improving, |
| | | | | | | | look |
| | | | | | | | good |
| 12 | 4.4 | S | ++ D | IBD, LI | + | 7 | Changed |
| | | | | | | | to |
| | | | | | | | probiotics |
| | | | | | | | only, |
| | | | | | | | Improving, |
| | | | | | | | look |
| | | | | | | | good |
| 2 | 3.9 | C | F. Megacolon | IBD, LI | + | 10 | Changed |
| | | | | | | | to |
| | | | | | | | probiotics |
| | | | | | | | only, |
| | | | | | | | Improving, |
| | | | | | | | look |
| | | | | | | | good |
| 2 | 3 | S | + D | IBD, LI | — | 3 | Changed |
| | | | | | | | to |
| | | | | | | | probiotics |
| | | | | | | | only, |
| | | | | | | | Improving, |
| | | | | | | | look |
| | | | | | | | good |

¹M, male; F, female; C, male, neuter; S: female, spay
²D: Diarrhea, O: bad month or body odor, V: Vomiting, C: Constipation, F: Flatulence, LA: Loss of Appetite

Table 6 shows the beneficial effects of probiotics on dogs with chronic biological change like inflammatory bowel diseases. The results show P. acidilactici and S. boulardii can be treated together with steroids to alleviate the symptoms.

Stability of Pediococcus acidilactici fermentative cultures mixed with Plant powders other than tomato powders.

Lemon Dried Powders

Pediococcus acidilactici fermentative cultures mixed with lemon powders, encapsulated in gelatin capsules and stored the gelatin capsules with the mixtures of lemon powders and P. acidilactici fermentative cultures at room temperature (varied from 18 C to 30 C). Ratio between lemon powder and P. acidilactici fermentative cultures by weight is Lemon Powder:P. acidilactici fermentative cultures=4:1

TABLE 7
Stability of encapsulation of P. acidilactici fermentative culture
with lemon dried powder in a gelatin capsule at room temperature.

| | Room temperature | |
|---|---|---|
| Date | (×10¹⁰ cfu/g)* | % of viable cells |
| 0 days | 1.1 | 100 |
| 19 days | 1.9 | 172 |
| 55 days | 2.9 | 262 |
| 304 days | 2.1 | 191 |
| 468 days | 0.9 | 82 |

*Separate the capsules and weight 0.5 g of the mixture of lemon powder and P. acidilactici fermentative cultures to resuspended in 0.5 ml PBS buffer, performed series of dilutions and plated 100 ul of diluted samples in MRS agar plates and incubated at 37 C. overnight in order to count numbers of viable cells.

Peach Powders

Pediococcus acidilactici fermentative cultures mixed with peach powders, encapsulated in gelatin capsules and stored the gelatin capsules with the mixtures of lemon powders and P. acidilactici fermentative cultures at room temperature (varied from 18 C to 30 C). Ratio between peach powder and P. acidilactici fermentative cultures by weight is peach powder:P. acidilactici fermentative cultures=4:1

TABLE 8
Stability of encapsulation of P. acidilactici fermentative culture
with peach dried powder in a gelatin capsule at room temperature.

| | Room temperature | |
|---|---|---|
| Days | (×10¹⁰ CFU/g) | % viable cells |
| 0 | 1.1 | 100 |
| 7 | 1.3 | 118 |
| 14 | 1.0 | 91 |
| 24 | 0.9 | 82 |
| 35 | 0.7 | 64 |
| 53 | 1.2 | 109 |
| 60 | 0.8 | 73 |
| 95 | 1.0 | 91 |
| 409 | 0.7 | 64 |
| 570 | 0.6 | 55 |

* Separate the capsules and weight 0.5 g of the mixture of peach powder and P. acidilactici fermentative cultures to resuspended in 0.5 ml PBS buffer, performed series of dilutions and plated 100 ul of diluted samples in MRS agar plates and incubated at 37 C. overnight in order to count numbers of viable cells.

Cabbage Powders Pediococcus

acidilactici fermentative cultures mixed with cabbage powders, encapsulated in gelatin capsules and stored at room temperature and stored the gelatin capsules with the mixtures of cabbage powders and P. acidilactici fermentative cultures at room temperature (varied from 18 C to 30 C). Ratio between cabbage powder and P. acidilactici fermentative cultures by weight is cabbage powder:P. acidilactici fermentative cultures=4:1

TABLE 9
Stability of encapsulation of P. acidilactici fermentative culture
with cabbage dried powder in a gelatin capsule at room temperature.

| | Room temperature × 10¹⁰ | |
|---|---|---|
| Date | (CFU/g) | % viable cells |
| 0 days | 4.6 | 100 |
| 4 days | 4.2 | 91 |
| 160 days | 1.4 | 30 |
| 463 days | 1.1 | 24 |

* Separate the capsules and weight 0.5 g of the mixture of cabbage powder and P. acidilactici fermentative cultures to resuspended in 0.5 ml PBS buffer, performed series of dilutions and plated 100 ul of diluted samples in MRS agar plates and incubated at 37 C. overnight in order to count numbers of viable cells.

Stability of Pediococcus acidilactici fermentative cultures mixed with Plant powders at different ratios.

Pediococcus acidilactici fermentative cultures mixed with tomato powders, encapsulated in gelatin capsules and store the gelatin capsules with the mixtures of tomato powders and P. acidilactici fermentative cultures at room temperature (varied from 18 C to 30 C) Ratio between tomato powder and P. acidilactici fermentative cultures by weight is Tomato Powder:P. acidilactici fermentative cultures=1:5

TABLE 10
Stability of encapsulation of P. acidilactici fermentative culture
with tomato dried powder in a gelatin capsule at room temperature.

| | Room temperature × 10¹⁰ | |
|---|---|---|
| days | (CFU/g) | % viable cells |
| 0 | 9.3 | 100 |
| 7 | 7.6 | 82 |
| 14 | 8.0 | 86 |
| 23 | 7.7 | 83 |
| 56 | 6.5 | 70 |
| 91 | 9.5 | 102 |
| 127 | 10.4 | 112 |
| 368 | 3.2 | 34 |
| 612 | 3.5 | 38 |
| 705 | 3.2 | 34 |

* Separate the capsules and weight 0.5 g of the mixture of tomato powder and P. acidilactici fermentative cultures to resuspended in 0.5 ml PBS buffer, performed series of dilutions and plated 100 ul of diluted samples in MRS agar plates and incubated at 37 C. overnight in order to count numbers of viable cells.

Stability of Pediococcus acidilactici fermentative cultures mixed with Saccharomyces boulardii and plant powders other than tomato powders.

Pediococcus acidilactici fermentative cultures mixed with S. boulardii fermentative cultures and Acerola powders, encapsulated in gelatin capsules. Store the gelatin capsules with the mixtures of P. acidilactici fermentative cultures, S. boulardii fermentative cultures, and Acerola powder at room temperature (varied from 18 C to 30 C)

Ratio between cabbage powder, P. acidilactici fermentative cultures and S. boulardii fermentative cultures by weight is P. acidilactici fermentative cultures:S. boulardii fermentative cultures:Acerola powder=0.4:0.3:2.5

TABLE 11
Stability of encapsulation of P. acidilactici fermentative
culture with S. boulardii fermentative cultures, and Acerola
dried powder in a gelatin capsule at room temperature

| | Room temperature × 10¹⁰ | |
|---|---|---|
| Date (days) | (CFU/g)* | % viable cells |
| 0 | 4.1 | 100 |
| 3 | 3.8 | 93 |
| 12 | 1.8 | 44 |
| 102 | 3.2 | 78 |

* Separated the capsules and weighted 0.5 g of the mixture of Acerola powder, P. acidilactici fermentative cultures and S. boulardii fermentative cultures, resuspended the mixtures in 0.5 ml PBS buffer. Performed series of dilutions, plated 100 ul of diluted samples in MRS agar plates and incubated at 37 C. overnight in order to count numbers of viable cells.

Stability of Pediococcus acidilactici fermentative cultures mixed with Plant powders stored in refrigerator: Pediococcus acidilactici fermentative cultures mixed with tomato powders, encapsulated in gelatin capsules and store the gelatin capsules with the mixtures of tomato powders and P. aicidilactici fermentative cultures at refrigerator (temperature varied from 4 C to 8 C). Ratio between tomato powder and P. acidilactici fermentative cultures by weight is Tomato Powder:P. acidilactici fermentative cultures=1:5

TABLE 12
Stability of encapsulation of P. acidilactici fermentative culture
with tomato dried powder in a gelatin capsule at refrigerator.

| | Refrigerator × 10¹⁰ | |
|---|---|---|
| days | (CFU/g) | % viable cells |
| 0 | 1.2 | 100 |
| 7 | 1.3 | 108 |
| 14 | 1.2 | 100 |
| 23 | 1.4 | 117 |
| 56 | 1.0 | 83 |
| 91 | 1.4 | 117 |
| 127 | 1.1 | 92 |
| 368 | 0.6 | 50 |
| 612 | 0.6 | 50 |
| 705 | 0.3 | 25 |

* Separate the capsules and weighted 0.5 g of the mixture of tomato powder and P. acidilactici fermentative cultures to resuspended in 0.5 ml PBS buffer, performed series of dilutions and plated 100 ul of diluted samples in MRS agar plates and incubated at 37 C. overnight in order to count numbers of viable cells.

Stability in mixture powders of microbial fermentative cultures with Acerola powders and stored at room temperature: Pediococcus acidilactici fermentative cultures mixed with S. boulardii fermentative cultures and Acerola powders. Store the powder mixtures of P. acidilactici fermentative cultures, S. boulardii fermentative cultures, and Acerola powder at room temperature (varied from 18 C to 30 C)

Ratio between Acerola powder, P. acidilactici fermentative cultures and S. boulardii fermentative cultures by weight is P. acidilactici fermentative cultures:S. boulardii fermentative cultures:Acerola powder=1.4:0.3:1.5 as well as P. acidilactici fermentative cultures:S. boulardii fermentative cultures:Acerola=0.4:0.3:2.5

TABLE 13
Stability of mixture powders of P. acidilactici fermentative culture
with S. boulardii fermentative cultures, and Acerola dried powder
at the ratio of 1.4:0.3:1.5 and stored at room temperature

| | Room temperature × 10¹⁰ | |
|---|---|---|
| days | (cfu/g)* | % viable cells |
| 0 | 3.2 | 100 |
| 33 | 2.2 | 67 |
| 60 | 2.8 | 88 |

*Weight 0.5 g of the mixture of P. acidilactici fermentative cultures, S. boulardii fermentative cultures and Acerola powder and resuspended in 0.5 ml PBS buffer, performed series of dilutions and plated 100 ul of diluted samples in MRS agar plates and incubated at 37 C. overnight in order to count numbers of viable cells. The ratio of mixture powders is - P. acidilactici fermentative cultures:S. boulardii fermentative cultures:Acerola dried powders = 1.4:0.3:1.5

TABLE 14
Stability of mixture powders of P. acidilactici fermentative culture
with S. boulardii fermentative cultures, and Acerola dried powder
at the ratio of 0.4:0.3:2.5 and stored at room temperature

| | Room temperature × 10¹⁰ | |
|---|---|---|
| days | (cfu/g)* | % viable cells |
| 0 | 4.1 | 100 |
| 120 | 4.2 | 102 |

*Weight 0.5 g of the mixture of P. acidilactici fermentative cultures, S. boulardii fermentative cultures and Acerola powder and resuspended in 0.5 ml PBS buffer, performed series of dilutions and plated 100 ul of diluted samples in MRS agar plates and incubated at 37 C. overnight in order to count numbers of viable cells. The ratio of mixture powders is - P. acidilactici fermentative cultures:S. boulardii fermentative cultures:Acerola dried powders = 0.4:0.3:2.5.

From these stability experiments about the encapsulated mixtures of P. acidilactici fermentative culture with different plant powders and microbial fermentative cultures in gelatin capsules, we have demonstrated the viable P. acidilactici was able to maintain greater than 34% viable cells from starting materials as stored at room temperature for more than 700 days, and the viable cells were greater than 25% as stored st at refrigerator.

Furthermore, it is demonstrated:

(1) P. acidilactici fermentative cultures are able to mix with dried plant powders such as peach powder, lemon powder, cabbage powder, tomato powder, Acerola powder.

(2) P. acidilactici fermentative cultures can be mixed with plant powders at different ratios: P. acidilactici fermentative cultures:plant (cabbage, peach, lemon) powders=1:4. P. acidilactici fermentative cultures:plant (tomato) powders=5:1, P. acidilactici fermentative cultures:plant (Acerola) powders=0.4:2.5, P. acidilactici fermentative cultures:plant (Acerola) powders=1.4:1.5.

(3) P. acidilactici fermentative cultures can be mixed with different microbial fermentative cultures (S. boulardii fermentative cultures)

(4) P. acidilactici fermentative cultures mixed with plant powders and microbial fermentative cultures are stable either in mixture powders or encapsulated in a gelatin capsule.

While a number of exemplary aspects and embodiments have been discussed above, those of skill in the art will recognize certain modifications, permutations, additions and subcombinations thereof. It is therefore intended that the following appended claims and claims hereafter introduced are interpreted to include all such modifications, permutations, additions and sub-combinations as are within their true spirit and scope. The applicant or applicants have attempted to disclose all the embodiments of the invention that could be reasonably foreseen. There may be unforeseeable insubstantial modifications that remain as equivalents.

1. A dry, stable and viable probiotic composition comprising,
a probiotic microorganism and
a dried plant powder, with the proviso that said composition is not a blended mixture of (1) at least one biologically pure Pediococcus acidilactici probiotic culture and (2) dried tomato powder at a weight ratio of 1:4, said blended mixture of (1) and (2) being encapsulated in an effective amount in a gelatin capsule.

2. The composition of claim 1, wherein the probiotic microorganism is selected from the group consisting of Pediococcus, Bifidobacterium, Bacteroides, Propionibacterium, Streptococcus, Enterococcus, Lactococcus, Lactobacillus, and Saccharomyces.

3. The composition of claim 1, wherein the dried plant powder is selected from the group consisting of dried vegetable powder, dried fruit powder, dried cereal powder, and dried herb powder.

4. The composition of claim 1, wherein the probiotic composition is encapsulated in a gelatin capsule.

5. The composition of claim 1, wherein the probiotic microorganism is Pediococcus or Saccharomyces.

6. The composition of claim 1, wherein the probiotic microorganism is Pediococcus acidilactici or Saccharomyces boulardii.

7. The process of ameliorating effects caused by environmental or biological changes in humans or animals comprising the step of:
feeding the human or animal in need of such amelioration an encapsulated probiotic composition comprising a viable encapsulated probiotic microbe, with the proviso that said composition is not a blended mixture of at least one biologically pure Pediococcus acidilactici probiotic culture and dried tomato powder at a weight ratio of 1:4 encapsulated in an effective amount in a gelatin capsule.

8. The process of claim 7, wherein the probiotic microorganism is selected from the group consisting of Pediococcus, Bifidobacterium, Bacteroides, Propionibacterium, Streptococcus, Enterococcus, Lactococcus, Lactobacillus, and Saccharomyces.

9. The process of claim 7, wherein the probiotic microorganism is Pediococcus or Saccharomyces.

10. The process of claim 7, wherein the probiotic microorganism is Pediococcus acidilactici or Saccharomyces boulardii.

11. The process of claim 7 wherein the animals are dogs or fish.

12. The process of ameliorating effects caused by environmental or biological changes in human or animals comprising the step:
feeding the human or animal in need of treatment for environmental or biological change or amelioration an encapsulated probiotic composition comprising:
a probiotic microorganism and
a dried plant powder, with the proviso that said composition is not a blended mixture of at least one biologically pure Pediococcus acidilactici probiotic culture and dried tomato powder at a weight ratio of 1:4 encapsulated in an effective amount in a gelatin capsule.

13. The process of claim 12, wherein the probiotic microorganism is selected from the group consisting of Pediococcus, Bifidobacterium, Bacteroides, Propionibacterium, Streptococcus, Enterococcus, Lactococcus, Lactobacillus, and Saccharomyces.

14. The process of claim 12, wherein the probiotic microorganism is Pediococcus or Saccharomyces.

15. The process of claim 12, wherein the probiotic microorganism is Pediococcus acidilactici or Saccharomyces boulardii.

16. The process of claim 12, wherein the dried plant powder is selected from the group consisting of dried vegetable powder, dried fruit powder, dried cereal powder, and dried herb powder.

17. The process of claim 12 wherein the animal is a dog or a fish.

18. The process of claim 7 wherein the biological change of human or animal is human or animal with Inflammatory Bowel Diseases (IBD).
